(19) United States
(12) Patent Application Publication
(10) Pub. No.: US 2012/0184739 A1
(43) Pub. Date: Jul. 19, 2012

(54) ENANTIOMERIC RESOLUTION OF 2,4-DISUBSTITUTED 1,3-OXATHIOLANE NUCLEOSIDES

(75) Inventors: Dragos Vizititu, Edmonton (CA); Ioana Simion, Edmonton (CA); Jean Eric Lacoste, Laval (CA)

(73) Assignee: SHIRE CANADA INC., VILLE SAINT-LAURENT, QUEBEC (CA)

(21) Appl. No.: 13/062,236

(22) PCT Filed: Sep. 5, 2008
(86) PCT No.: PCT/IB2008/002314

§ 371 Date: Jan. 17, 2012

(51) Int. Cl. C07D 411/04

(52) U.S. Cl. 544/317

(57) ABSTRACT

Single enantionmers of compounds of formula (B), in either the cis or trans configuration,

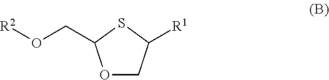

wherein R¹ and R² are as defined herein, can be separated from enantiomeric mixtures thereof by reacting the compound with an acid to produce a conglomerate salt that has the following characteristics:
the IR spectrum of the salt of the racemic compound, a 1:1 mixture of (−) and (+) crystals, is identical to that of the each of the single enantiomer, and
the salt of the racemic compound has a melting point lower that that of either single enantiomer. The conglomerate salt is then separated by preferential crystallization.

### FIELD OF THE INVENTION

The present invention relates to a novel process for producing (−) and (+) isomers of cis nucleosides or nucleoside analogues and derivatives of formula (A):

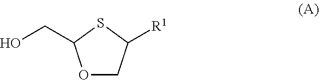

wherein R¹ is a pyrimidine base or a pharmaceutically acceptable derivative thereof.

### BACKGROUND OF THE INVENTION

Classes of compounds of formula (A), particularly the 2,4-disubstituted 1,3-oxathiolanes pyrimidine nucleosides and derivatives thereof, have been found to have potent antiviral activity. In particular, these compounds have been found to act as potent inhibitors of HIV-1 replication in T-lymphocytes over a prolonged period of time with less cytotoxic side effects than compounds known in the art (see Belleau et al (1993) Bioorg. Med. Chem. Lett. Vol. 3, No. 8, pp. 1723-1728). These compounds have also been found active against 3TC-resistant HIV strains (see Taylor et al (2000) Antiviral Chem. Chemother. Vol 11, No. 4, pp. 291-301; Stoddart et al (2000) Antimicrob. Agents Chemother. Vol. 44, No. 3, pp. 783-786). Additionally, the compounds of formula (A) are also useful in prophylaxis and treatment of hepatitis B virus infections.

Methods for the preparation of these compounds have been disclosed in PCT publications WO 92/08717, WO 95/29176 and WO 02/102796, as well as in publications by Belleau et al (1993) Bioorg. Med. Chem. Lett. Vol. 3, No. 8, pp. 1723-1728; Wang et al (1994) Tetrahedron Lett. Vol. 35, No. 27, pp. 4739-4742; Mansour et al , (1995) J. of Med. Chem. Vol. 38, No. 1, pp. 1-4 and Caputo et al in Eur. J. Org. Chem. Vol. 6, pp. 1455-1458 (1999).

The products of these processes are in many cases a racemate. These racemates require further processing to obtain the pure enantiomers. A preferred method for the production of single enantiomers is resolution of a racemate such as by direct preferential crystallization, crystallization of the diastereomeric salts, kinetic resolution, enzymatic resolution, selective absorption and asymmetric synthesis. See, e.g., EP 0 515 156, EP 0 515 157, EP 0560 794, EP 0 756 595, EP 0 757 684, EP 1 153 924, EP 1 361 227, EP 1 406 896, EP 1 473 294, EP 1 632 490, U.S. Pat. No. 5,663,320, U.S. Pat. No. 5,693,787, U.S. Pat. No. 6,600,044, US 2006/0199786, WO 92/20669, WO 92/20696, and WO 2006/096954.

For example, Cimpoia et al. (US 2006/0199786) discloses a method preparing optically active cis-2-hydroxymethyl-4-(cytosine-1′-yl)-1,3-oxathiolane and derivatives thereof by reacting cis-oxathiolane compound with a chiral acid to for two diastereomeric salts, recovering one of the diastereomeric salts, and converting the recovered diastereomeric salt back into a enantiomer of the base compound.

If the racemate is a “true” racemic compound, a homogeneous solid phase of the two enantiomers co-exists in the same cell unit. These materials may be separated via diastereomer crystallization, which generally involves reacting the racemate with an optically pure acid or base (i.e., a resolving agent) to form a mixture of diastereomeric salts. These mixtures may be separated by preferential crystallization. However, some racemates may exist in the form of conglomerates. In a conglomerate, the individual enantiomers each crystallize as a single crystal lattice. Thus, a conglomerate salt is in effect a physical mixture of two separate crystal types, one of each isomer. But, conglomerates are typically observed in less than 20% of all racemates. See, e.g., Lorenz, H., et al., J. of the Univ. of Chem. Tech. and Metallurgy, (2007), 42 (1), 5-16 [5 to 10% of racemates belong to the conglomerate forming group].

A conglomerate can be defined as an equimolar mixture of two crystalline enantiomers that are, in principle, mechanically separable. The phase diagram of a conglomerate displays one sharply defined minimum temperature at a mixture of 50% and 50% that is the eutectic point of the enantiomeric mixture. The success of a preferential crystallization depends on this fact.

Methods for resolving certain racemates by formation of conglomerate salts, also known as preferential crystallization or resolution by entrainment, are described in, for example, Tung et al. (U.S. Pat. No. 4,994,604), Manimaran et al. (U.S. Pat. No. 5,302,751), and Coquerel et al. (U.S. Pat. No. 6,022,409), the entire disclosures of which are hereby incorporated by reference.

A conglomerate compound crystallizes as a single enantiomer in the crystal lattice, i.e., each crystal lattice is made up of a single enantiomer. Therefore, to be a conglomerate, the IR spectrum of the racemic conglomerate salt, a 1:1 mixture of (−) and (+) crystals, must be identical to that of the single enantiomer. Another characteristic of conglomerate behavior is that the racemic conglomerate salt normally has a melting point lower that that of either single enantiomer.

If a conglomerate is obtained, it may be used for enantiomeric excess enhancement because the most soluble composition is racemic. Generally, if the conglomerate has an excess of one enantiomer, that excess can be recovered, i.e., the conglomerate of X % enantiomeric excess will provide an X % yield of single enantiomer leaving behind racemic liquors.

A conglomerate in racemic form may also be used in an entrainment process in which a racemic solution is seeded with a single enantiomer leading to preferential kinetic precipitation of that enantiomer. See, e.g., Lorenz, H., et al., J. of the Univ. of Chem. Tech. and Metallurgy, (2007), 42 (1), 5-16.

### SUMMARY OF THE INVENTION

While procedures as described above offer effective means to obtain single isomers of the cis nucleoside or nucleoside analogues and derivatives of formula (A):

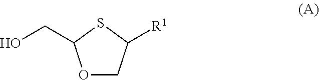

wherein R¹ is a pyrimidine base or a pharmaceutically acceptable derivative thereof, there is a need for a simpler and more economical process. The present invention is based on the discovery of a process which permits the enantiomers to be separated directly and efficiently by a direct crystallization technique using specific conglomerate salts.

Thus, according to a process aspect of the present invention, there is provided a method for the preparation of single enantiomers of compounds of formula (B) in the cis configuration, and pharmaceutically acceptable salts and esters thereof,

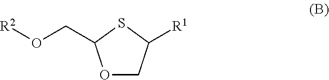

wherein
R¹ is pyrimidine base or a pharmaceutically acceptable derivative thereof,
R² is hydrogen, a carboxyl function —C(O)—R³, or together with the oxygen atom to which it is attached forms an ester derived from a polyfunctional acid (such as phosphoric acids or carboxylic acids containing more than one carboxyl group, e.g., dicarboxylic acids of the formula HO₂C(CH₂)₁₋₁₀CO₂H); and
R³ is selected from hydrogen, straight or branched chain (e.g., methyl, ethyl, n-propyl, t-butyl, n-butyl) or cyclic alkyl having 1 to 30 carbon atoms which is unsubstituted or substituted, alkoxyalkyl (e.g., methoxymethyl) having 2 to 30 carbon atoms which is unsubstituted or substituted, aralkyl (e.g., benzyl) having 7 to 18 carbon atoms which is unsubstituted or substituted, aryloxyalkyl (e.g., phenoxymethyl) having 7 to 18 carbon atoms which is unsubstituted or substituted, aryl having 6 to 14 carbon atoms which is unsubstituted or substituted (e.g., phenyl optionally substituted by halogen, C₁₋₄ alkyl or C₁₋₄ alkoxy), substituted dihydropyridinyl (e.g., N-methyldihydropyridinyl), sulphonate esters such as C₁₋₆-alkyl- or C₇₋₁₈-aralkylsulphonyl (e.g., methanesulphonyl), sulfate esters, amino acid esters (e.g., L-valyl or L-isoleucyl) and mono, di- or triphosphate esters,
   the process comprising:
forming a conglomerate salt of a racemic mixture or an enantiomerically enriched mixture of a compound of formula (B) with an acid, wherein the resulting conglomerate salt has the following characteristics:
   the IR spectrum of the salt of the racemic compound, a 1:1 mixture of (−) and (+) crystals, is identical to that of each of the single enantiomer, and
   the salt of the racemic compound has a melting point lower that that of either single enantiomer;
obtaining an enantiomerically enriched mixture of the salts of the enantiomers by crystallization; and
obtaining the free base of the enantiomerically enriched mixture by standard methods (i.e., converting the salt into the free base).

Following formation of the conglomerate salt, the enantiomers can be separated by preferential crystallization such as described in Tung et al. (U.S. Pat. No. 4,994,604), Manimaran et al. (U.S. Pat. No. 5,302,751), and Coquerel et al. (U.S. Pat. No. 6,022,409). The enantiomers may also be separated by a process of entrainment 15 or cyclic entrainment.

In the present invention a solution of the cis nucleoside of formula B may be entrained by seeding with crystals of the desired single enantiomer to grow larger crystals having an excess of the isomer seeded, and leaving the opposite isomer enriched in the mother liquors. For an entrainment crystallization procedure to be useful for the production of single-enantiomer cis nucleoside of formula B, it is desirable that the enantiomerically enriched nucleoside obtained can be raised in enantiomeric purity through recrystallization or a series of recrystallizations. The mother liquors enriched with the opposite isomer may be treated further. The opposite isomer may be extracted via a similar recrystallization or it could be racemized, and the seeding process described above repeated allowing all the material in the mother liquor to be directed to the required cis isomer. Therefore, the process object of the present invention would provide crystals of higher enantiomeric excess (ee) of the desired isomer of the cis nucleoside of formula B. This would allow the present invention to be combined with methods which initially produce the cis nucleoside of formula B crystals of low ee, (such as a method of asymmetric synthesis that produces material of unacceptable ee) to provide a final product having a much higher ee of the desired product.

This process may also be used to prepare the single enantiomers of compounds of formula (B) in the trans configuration.

In an alternative embodiment of the present invention, the conglomerate salt of cis 2′-deoxy-3′-oxa-4′-thiocytidine is formed, wherein the single enantiomer shows a much lower solubility than the racemate in polar solvents.

The present invention includes the direct enantiomer separation of enantiomeric mixtures of cis 2′-deoxy-3′-oxa-4′-thiocytidine or cis/trans combinations of 2′-deoxy-3′-oxa-4′-thiocytidine without the need for resolving agents, by seeding a supersaturated solution of the 2′-deoxy-3′-oxa-4′-thiocytidine conglomerate salt with the desired single enantiomer 2′-deoxy-3′-oxa-4′-thiocytidine conglomerate salt, under controlled conditions.

The present invention also includes a process for the preparation of a single enantiomer of a compound of formula (B) or a pharmaceutically acceptable salt or ester thereof, wherein the enantiomer comprises methyl tosylate in an amount equal to or less than 2 ppm, the process comprising:
(a) forming a conglomerate salt of a racemic mixture or an enantiomerically enriched mixture of a compound of formula (B) with a tosic acid;
(b) obtaining an enantiomerically enriched mixture of the salts of the enantiomers by crystallization; and
(c) obtaining the free base of the enantiomerically enriched mixture, wherein the enantiomerically enriched mixture contains methyl tosylate in an amount equal to or less than 2 ppm.

The present invention also includes a composition comprising a single enantiomer of 2′-deoxy-3′-oxa-4′-thiocytidine or a pharmaceutically acceptable salt or ester thereof, wherein the enantiomer comprises methyl tosylate in an amount equal to or less than 2 ppm.

### DETAILED DESCRIPTION OF THE INVENTION

Accordingly, there is a provided in a first aspect of this invention the preparation of a single enantiomer of compounds of formula (B) in the cis configuration

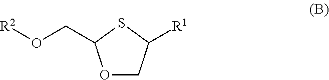

wherein R¹ and R² are as defined above, and pharmaceutically acceptable salts and esters thereof, via the formation of a conglomerate salt of a racemic mixture or an enantiomerically enriched mixture of a compound of formula (B) with an acid wherein the resulting conglomerate salt has the following characteristics: an IR spectrum of the salt of the racemic compound, a 1:1 mixture of (−) and (+) crystals, which is identical to each of the single enantiomer, and the salt of the racemic compound has a melting point lower that that of either single enantiomer.

In a preferred embodiment the single enantiomer further comprises a second isomer of a compound of formula (B) in an amount equal to or less than 1%. For example, in the case that the single enantiomer is the (−) cis isomer it will be understood that the second isomer may be selected from the (+) cis isomer, the (−) trans isomer, the (+) trans isomer and mixtures thereof.

The acid is preferably selected from maleic acid, achiral acids such as tosic acid, and mixtures thereof.

The present invention is based on the formation of a conglomerate salt of 2-substituted 4-substituted 1,3-oxathiolanes of formula (B) wherein R¹ is pyrimidine base or a pharmaceutically acceptable derivative thereof and R² is hydrogen, or together with the oxygen atom to which it is attached forms an ester of a polyfunctional acid, or a carboxyl function —C(O)—R³ in which the non-carbonyl moiety R³ of the ester grouping is selected from hydrogen, straight or branched chain alkyl (e.g., methyl, ethyl, n-propyl, t-butyl, n-butyl), C₃₋₈ cyclic alkyl, alkoxyalkyl (e.g., methoxymethyl), aralkyl (e.g., benzyl), aryloxyalkyl (e.g., phenoxymethyl), aryl (e.g., phenyl optionally substituted by halogen, C₁₋₄ alkyl or C₁₋₄ alkoxy); substituted dihydropyridinyl (e.g., N-methyldihydropyridinyl), sulphonate esters such as alkyl- or aralkylsulphonyl (e.g., methanesulphonyl), sulfate esters, amino acid esters (e.g., L-valyl or L-isoleucyl) and mono, di- or triphosphate esters.

Preferably, R¹ is selected from the following formulae:

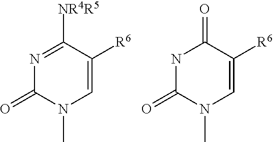

wherein

R⁴ and R⁵ are in each case independently H, straight, branched or cyclic C₁₋₆ alkyl, straight, branched or cyclic C₂₋₆ alkenyl, C₆₋₁₄ aryl, or C₅₋₁₀ heteroaromatic ring containing 1-3 heteroatoms wherein each heteroatom is O, N, or S heteroatoms; and

R⁶ is hydrogen, hydroxymethyl, trifluoromethyl, straight, branched or cyclic C₁₋₆ alkyl, straight, branched or cyclic C₂₋₆ alkenyl, bromine, chlorine, fluorine, or iodine.

R¹ may be, for example, cytosine or 5-fluorocytosine.

R² also includes esters derived from polyfunctional acids such as carboxylic acids containing more than one carboxyl group, for example, dicarboxylic acids HO₂C(CH₂)ₙCO₂H where n is an integer of 1 to 10 (for example, succinic acid) or phosphoric acids. For example, R² can be of the formula HO₂C(CH₂)ₙCO—O— where n is 1 to 10. Methods for preparing such esters are well known. See, for example, E. Hahn et al., “Nucleotide dimers as anti-human immunodeficiency virus agents”, Nucleotide Analogues As Antiviral Agents, J. C. Martin, Ed. Symposium Series #401, American Chemical Society, pp. 156-159 (1989) and M. Busso et al., “Nucleotide dimers suppress HIV expression in vitro”, AIDS Research and Human Retroviruses, 4(6), pp. 449-455 (1988).

The present invention includes the formation of a conglomerate salt of cis 2′-deoxy-3′-oxa-4′-thiocytidine wherein the single enantiomer shows a much lower solubility than the racemate in polar solvents. Direct enantiomer separation, without the need for resolving agents, can be achieved by seeding a supersaturated solution of the racemate with a single enantiomer, under controlled conditions. The separation may also be achieved for any derivative thereof.

An embodiment of the present invention includes a method for resolving cis-2-hydroxymethyl-4-(cytosin-1′-yl)-1,3-oxathiolane or derivatives or salts thereof comprising:

a) reacting said cis-2-hydroxymethyl-4-(cytosin-1′-yl)-1,3-oxathiolane with an achiral acid to produce cis-2-hydroxymethyl-4-(cytosin-1′-yl)-1,3-oxathiolane•achiral acid salt;

b) preparing a solution of cis-2-hydroxymethyl-4-(cytosin-1′-yl)-1,3-oxathiolane•achiral acid salt having an enantiomeric excess greater than zero;

c) adding to said solution an amount of (+) or (−)-cis-2-hydroxymethyl-4-(cytosin-l′-yl)-1,3-oxathiolane•achiral acid salt sufficient to initiate crystallization;

d) recovering substantially one of said (+) or (−)-cis-2-hydroxymethyl-4-(cytosin-1′-yl)-1,3-oxathiolane•achiral acid salt; and

e) converting said (+) or (−)-cis-2-hydroxymethyl-4-(cytosin-l′-yl)-1,3-oxathiolane•achiral acid salt into said (+) or (−)-cis-2-hydroxymethyl-4-(cytosin-1′-yl)-1,3-oxathiolane or salts.

Examples of achiral acids useful in the formation of conglomerate salts include hydrochloric acid (HCl), hydrobromic acid (HBr), sulfuric acid (H₂SO₄), tetrafluoroboric acid (HBF₄), methanesulfonic acid (CH₃SO₃H), benzenesulfonic(BS) acid (C₆H₅SO₃H), p-toluenesulfonic acid (p-CH₃C₆H₄SO₃H), p-aminoBS acid (p-NH₂C₆H₄SO₃H), p-chloroBS acid (p-ClC₆H₄SO₃H), p-hydroxyBS acid (p-HOC₆H₄SO₃H), chloroacetic acid (ClCH₂COOH), dichloroacetic acid (Cl₂CHCOOH), trichloroacetic acid (Cl₃CHCOOH), glycolic acid (HOCH₂COOH), pyruvic acid (CH₃COCOOH), succinic acid (HOOC(CH₂)₂COOH), adipic acid, (HOOC(CH₂)₄COOH), maleic acid (Cis-HOOCCH═CHCOOH),fumaric acid (Tr-HOOCCH═CHCOOH) , citric acid (HOC(CO₂H)(CH₂CO₂H)₂), and mixtures thereof, among others.

In accordance with another aspect of the present invention there is provided a process for the preparation of a single enantiomer of a compound of formula (B) or a pharmaceutically acceptable salt or ester thereof, wherein the enantiomer comprises methyl tosylate in an amount equal to or less than 2 ppm, the process comprising the steps of:
(a) forming a conglomerate salt of a racemic mixture or an enantiomerically enriched mixture of a compound of formula (B) with a tosic acid;
(b) obtaining an enantiomerically enriched mixture of the salts of the enantiomers by crystallization; and
(c) obtaining the free base of the enantiomerically enriched mixture, wherein the enantiomerically enriched mixture contains methyl tosylate in an amount equal to or less than 2 ppm.

Preferably, the tosic acid is para-toluenesulfonic acid, the compound of formula (B) is 2′-deoxy-3′-oxa-4′-thiocytidine, and the enantiomer is in the cis configuration.

In accordance with still another aspect, the present invention provides a composition comprising a single enantiomer of 2′-deoxy-3′-oxa-4′-thiocytidine or a pharmaceutically acceptable salt or ester thereof, wherein the enantiomer comprises methyl tosylate in an amount equal to or less than 2 ppm. Preferably, the enantiomer is in the cis configuration.

By the term “ derivative” is a compound which is a pharmaceutically acceptable salt, ester, or salt of such ester of a compound of formula (B), or any other compound which, upon administration to the recipient, is capable of providing (directly or indirectly) a compound of formula (B) or an antivirally active metabolite or residue thereof. It will be appreciated by those skilled in the art that the compounds of formula (B) may be modified to provide pharmaceutically acceptable derivatives thereof, at functional groups in the base moiety.

The term “alkyl”, as used herein, unless otherwise specified, refers to a saturated straight, branched, or cyclic, primary, secondary, or tertiary hydrocarbon of having 1-30 carbon atoms, preferably 1-6 carbon atoms, which is unsubstituted or optionally mono- or di-substituted by hydroxy, N₃, CN, SH, amino, halogen (F, Cl, Br, I), C₆₋₁₂-aryl, C₁₋₆-alkyl, C₂₋₁₂-alkoxyalkyl, or nitro. It specifically includes methyl, ethyl, cyclopropyl, propyl, isopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl.

Thus, R² can be, for example, methyl, ethyl, propyl, isopropyl, butyl, isobutyl, t-butyl, pentyl, isopentyl, neopentyl, hexyl, isohexyl, cyclopropyl, cyclobutyl, cyclopentyl, cyclohexyl, cyclopropylmethyl, cyclopentylmethyl, cyclohexylmethyl, 3-methylpentyl, 2,2-dimethylbutyl, 2,3-dimethylbutyl, halogenated C₁₋₆-alkyl, C₁₋₆-hydroxyalkyl, or C₁₋₆-aminoalkyl.

The term “alkenyl”, as used herein, unless otherwise specified, represents an alkyl radical as defined herein wherein one or more —CH₂—CH₂— groups is in each case replaced by —CH═CH—. The alkenyl groups can be substituted in the manner described above for alkyl groups.

Alkoxyalkyl, as used herein, refers to alkyl-O-alkyl groups having up to 30 carbon atoms, preferably up to 6 carbon atoms, which in each case is unsubstituted or optionally mono- or di-substituted by hydroxy, N₃, CN, SH, amino, or halogen (F, Cl, Br, I). It specifically includes methoxymethyl, ethoxymethyl, propoxymethyl, and butoxymethyl.

The term “aryl” represents an aromatic moiety which is unsubstituted or substituted one or more times by hydroxy, N₃, CN, C₁₋₄ alkyl, C₁₋₄ alkoxy, and/or halogen (F, Cl, Br, I) and containing at least one benzenoid-type ring. The aryl groups contain from 6 to 14 carbon atoms (e.g., phenyl and naphthyl), particularly 6 to 10 carbon atoms.

The term “aralkyl” represents an aryl moiety which is attached to the adjacent atom by an alkyl group. The aryl portion of aralkyl is optionally substituted one or more times by hydroxy, N₃, CN, C₁₋₄ alkyl, C₁₋₄ alkoxy, and/or halogen (F, Cl, Br, I) and containing at least one benzenoid-type ring.

The term “aryloxyalkyl” represents an aryl moiety which is attached to an alkyl group by an oxygen atom, i.e., aryl-O-alkyl. The aryl portion of aryloxyalkyl is optionally substituted one or more times by hydroxy, N₃, CN, C₁₋₄ alkyl, C₁₋₄ alkoxy, and/or halogen (F, Cl, Br, I) and containing at least one benzenoid-type ring.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis. Suitable protecting groups are described, for example, in Greene, et al., “Protective Groups in Organic Synthesis,” John Wiley and Sons, Second Edition, 1991, hereby incorporated by reference.

According to an embodiment of the invention,

R¹ is selected from the following formulae:

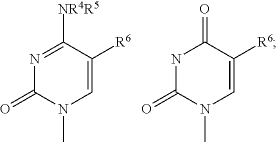

R² is —C(O)—R³;

R³ is methyl, ethyl, cyclopropyl, propyl, isopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, 2,3-dimethylbutyl, methoxymethyl, phenyl, phenyl which is substituted by halogen, C₁₋₄₆ alkyl, or C₁₋₄ alkoxy, benzyl, or phenoxymethyl;

R⁴ and R⁵ are in each case independently H, straight, branched or cyclic C₁₋₆ alkyl, straight, branched or cyclic C₂₋₆ alkenyl, C₆₋₁₄ aryl, or C₅₋₁₀ heteroaromatic ring containing 1-3 O, N, or S heteroatoms; and

R⁶ is hydrogen, hydroxymethyl, trifluoromethyl, straight, branched or cyclic C₁₋₆ alkyl, straight, branched or cyclic C₂₋₆ alkenyl, bromine, chlorine, fluorine, or iodine.

According to another embodiment of the invention,

R¹ is cytosine or 5-fluorocytosine;

R² is —C(O)—R³; and

R³ is methyl, ethyl, cyclopropyl, propyl, isopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, 2,3-dimethylbutyl, methoxymethyl, phenyl, phenyl which is substituted by halogen, C₁₋₄₆ alkyl, or C₁₋₄ alkoxy, benzyl, or phenoxymethyl.

The present invention includes the formation of crystalline cis 2′-deoxy-3′-oxa-4′-thiocytidine, enriched in the desired enantiomer, without requiring the use of seed crystals of desired enantiomer.

The present invention also includes the formation of crystalline cis 2′-deoxy-3′-oxa-4′-thiocytidine using a seed crystal of the desired enantiomer.

The present invention also includes the formation of crystalline cis 2′-deoxy-3′-oxa-4′-thiocytidine starting from cis/trans mixtures of 2′-deoxy-3′-oxa-4′-thiocytidine, wherein the cis to trans ratio is between about 1/1 to about 5/1.

The present invention also includes an entrainment process. Firstly, a saturated solution of the racemic cis 2′-deoxy-3′-oxa-4′-thiocytidine or a derivative is prepared at a given temperature. Of particular interest are solvents which favor the crystallization of the compound of formula (B). Suitable solvents include water, methanol, ethanol, toluene, tert-butyl methyl ether, isopropanol, n-propanol, acetone, and combinations thereof.

In an embodiment of the present invention, an amount of racemic mixture of cis 2′-deoxy-3′-oxa-4′-thiocytidine or a derivative thereof is dissolved or suspended in a suitable solvent. Heat may be used to complete the dissolution. Concentrations above the saturation point may be used. The conglomerate is formed by adding in excess maleic acid, to the solution or suspension of the 2′-deoxy-3′-oxa-4′-thiocytidine or a derivative thereof to form a salt. The amount of achiral acid used is greater than about 1 eq. The amount of achiral acid may be between about 1 and about 3 eq. The conglomerate salt may be crystallized by conventional means. The melting point of the conglomerate salt is about 20° C. lower than the melting point of the single enantiomer salt. The eutectic point of the para-toluenesulfonic acid salt of 2′-deoxy-3′-oxa-4′-thiocytidine is between about 185° C. and 187° C. The eutectic point of the maleic salt of 2′-deoxy-3′-oxa-4′-thiocytidine is between about 171° C. and 173° C.

Once the conglomerate salt is formed the reaction mixture may be seeded with crystals of the desired single enantiomer salt or the mixture may proceed to crystallization by conventional means. The seeded or unseeded mixture is then cooled and once crystallization has taken place, the precipitate product is harvested. The precipitate product shows a greater weight excess of desired single enantiomer salt. The mother liquor shows an excess of the enantiomer (opposite to that used for the seeding if seeding was used).

The precipitate product may be recrystallized by resuspending the precipitate product in a suitable recrystallization solvent. Suitable recrystallization solvents may include alcohols such as methanol, ethanol, isopropanol, acetone, and combinations thereof.

To obtain the free base, the precipitate product is resuspended in a suitable recrystallization solvent. Suitable recrystallization solvents include alcohols such as methanol, ethanol, isopropanol, acetone, and combinations thereof. If necessary, the pH is adjusted so that the mixture is basic (pH 7). A base is used to remove the acid. The base may be a free amine such as triethylamine, diethylcyclohexylamine, diethylmethylamine, dimethylethylamine, dimethylisopropylamine, dimethylbutylamine, dimethylcyclohexylamine, tributylamine, diethylmethylamine, dimethylisopropylamine, diisopropylethylamine or combinations thereof, or an immobilized base such as anion exchange resin or even ammonia. If a resin is used, the resin may be removed by filtration. The free base is cooled and the resulting precipitate is dried. The resultant crystalline cis 2′-deoxy-3′-oxa-4′-thiocytidine is enriched in the desired enantiomer. The amount of base added should be sufficient to remove all of the acid counter ions.

When a tosic acid such as para-toluene sulfonic acid is employed to form the conglomerate salt, the resultant enantiomer preferably comprises methyl tosylate in an amount equal to or less than 2 ppm.

The mother liquors resulting from the above described procedure contain an excess of one enantiomer that can be re-subjected to the above procedure by seeding with the opposite enantiomer. By an iterative process of crystallization (cyclic entrainment), seeding with opposite enantiomers alternately, it is, in principle, possible to separate an amount of racemic 2′-deoxy-3′-oxa-4′-thiocytidine entirely into its enantiomeric components.

In the enantiomeric enrichment (ee) procedure of this invention, the recrystallization may be preformed in a variety of solvents. These solvents can be chosen and the crystallization process induced by conventional techniques that lead to the formation of a supersaturated solution. Examples of such conventional techniques include cooling of a saturated solution, solvent evaporation from a saturated solution, or by employing a counter solvent in which the desired end product, such as cis-2′-deoxy-3′-oxa-4′-thiocytidine, is less soluble.

The present invention additionally includes the preparation of conglomerate salts described above using cis/trans mixtures of 2-substituted 4-substituted 1,3-oxathiolanes, wherein the cis to trans ratio (C/T) is between about 1/4 to about 4/1, for example, 1.6/1 to 3.5/1, especially 2/1 to 3/1.

In general, if an enantiomerically enriched mixture or a cis/trans combination (wherein C/T>1) of a compound of formula (B) is to be separated, the process may proceed through the following steps:1) formation of the conglomerate salt; 2) isolation of the enantiomerically enriched precipitate salt; 3) liberation of the enantiomerically enriched free base (the compound of formula (B)) from the precipitate salt by reaction of the salt with a proper base; 4) isolation of the enantiomerically enriched compound of formula (B) precipitate.

An embodiment of the present invention is a process for producing (−)-cis-2-hydroxymethyl-4-(cytosin-1′-yl)-1,3-oxathiolane, comprising:

a) preparing a solution of cis-2-hydroxymethyl-4-(cytosin-1′-yl)-1,3-oxathiolaneoachiral acid salt having an enantiomeric excess different than zero;

b) crystallizing substantially (−)-cis-2-hydroxymethyl-4-(cytosin-l′-yl)-1,3-oxathiolaneoachiral acid salt;

c) recovering said (−)-cis-2-hydroxymethyl-4-(cytosin-l′-yl)-1,3-oxathiolane•achiral acid salt;

d) converting said (−)-cis-2-hydroxymethyl-4-(cytosin-l′-yl)-1,3-oxathiolane•achiral acid salt into said (−)-cis-2-hydroxymethyl-4-(cytosin-1′-yl)-1,3-oxathiolane or pharmaceutically acceptable salts thereof.

Another embodiment of the present invention is the para-toluenesulfonic acid salt of 2′-deoxy-3′-oxa-4′-thiocytidine having an eutectic point between about 185° C. and 187° C.

Another embodiment of the present invention is the of the maleic salt of 2′-deoxy-3′-oxa-4′-thiocytidine having an eutectic point between about 171° C. and 173° C.

### BRIEF DESCRIPTION OF THE DRAWINGS

Various features and attendant advantages of the present invention will be more fully appreciated as the same becomes better understood when considered in conjunction with the accompanying drawings, wherein:

FIG. 1 is a phase diagram of the p-Toluenesulfonic acid salt of Compound (1); and

FIG. 2 illustrates the UV and optical rotation monitoring of the crystallization process shown in Example 2.

The entire disclosure of all applications, patents and publications, cited above and below, is hereby incorporated by reference.

### EXAMPLES

### Example 1

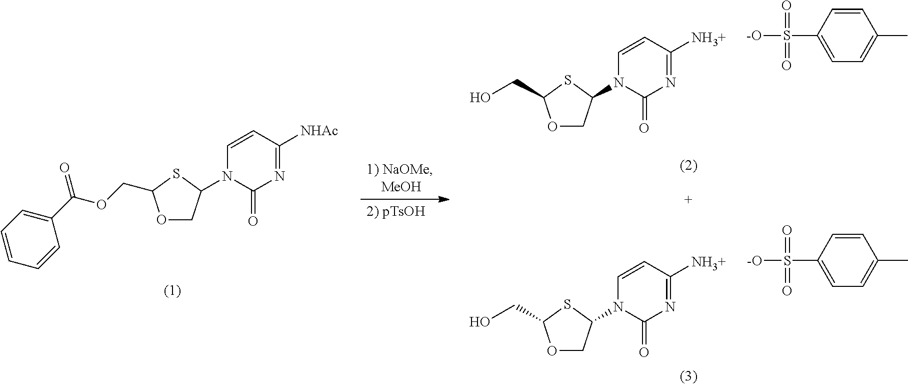

Compound (1) was prepared as described in PCT publication WO 02/102796. Sodium methoxide (0.1 eq.) was added in one portion to a methanol suspension (70 mL) of Compound 1 (1.0 eq.) at room temperature. The reaction mixture was stirred for 2 hrs at room temperature. TLC analysis (Hexane/Et0Ac:1/9) showed the disappearance of starting material and the appearance of the more polar deprotected (1). para-Toluenesulfonic acid (1.14 eq.) was added to the solution in one portion at room temperature. The reaction mixture was allowed to stir at room temperature overnight. The reaction mixture was cooled to 0° C.-5° C. The suspension was stirred at this temperature for 1 hour then filtered. The solids were dried to give pure Compounds (2) and (3) as a white solid.

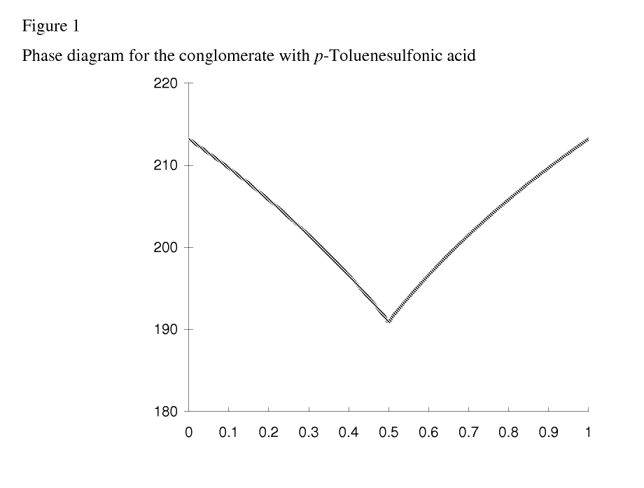

The p-TSA salts of both enantiomers and racemates were prepared and recrystallized from methanol/water/IPA. The maleic acid salt was obtained using the same solvent system. The IR spectra and Differential Scanning calorimetry (DSC) results are shown in Table 1, Table 2 and FIG. 1.

TABLE 1

| | |
|---|---|
| IR | DSC data (5° C./min) |

| p-TSA Salt | Match | Melting Point (° C.) | ΔH (J/g) |
|---|---|---|---|
| Racemate | Yes | 186.8 | 100.2 |
| (−) enantiomer | Yes | 213.2 | 145.6 |
| (+) enantiomer | Yes | 214.2 | |

The maleic acid salt was prepared in a similar fashion.

TABLE 2

| | |
|---|---|
| Maleic acid | DSC data (7° C./min) |

| Salt | IR Match | Melting Point (° C.) | ΔH (J/g) |
|---|---|---|---|
| Racemate | Yes | 172.0 | — |
| Single | Yes | 238.8 | — |
| enantiomer | | | |

### Example 2

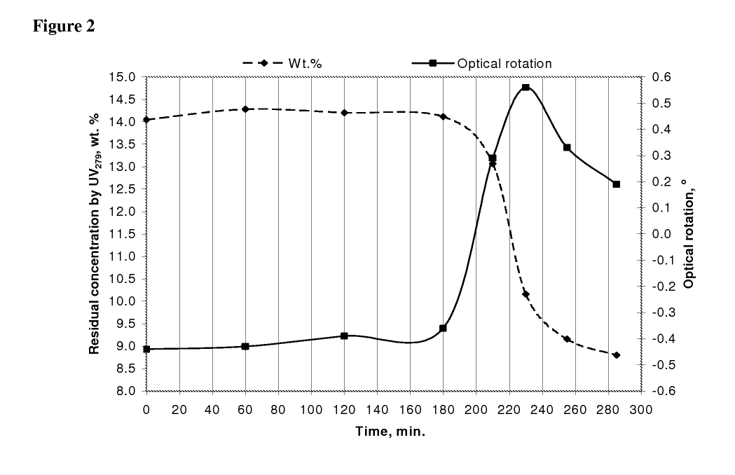

A 13 wt % racemate mixture of Compound (2) and (3) solution was prepared by dissolving 104.60 g of the racemate in 700 ml water. A 4% ee was generated by adding 4.36 g Compound (2) to the mixture. The solids were dissolved by heating the slurry at 50° C. The warm solution was cooled rapidly to 20° C. and then, agitated at this temperature for 1 more hour to ensure its stability. Next, the supersaturated solution was seeded with 202 mg of finely ground Compound (2) (25 mg/100 g solution). The temperature was maintained constant at 20±1° C. with constant agitation. The course of crystallization was monitored by UV at 278 nm and polarimetry (see FIG. 2 below).

The optical rotation of the starting supersaturated solution was −0.44°. A 3-hour induction period was recorded before the crystallization occurred. Approximately 20 minutes into crystallization, the rotation of the solution dropped to zero. Further, the rotation changed sign and reached the maximum of +0.56° after about 50 minutes of crystallization. Changing rotation sign of the supernatant solution indicates that entrainment and resolution has occurred. The solids were filtered out and the optical purity determined. The isolated solid had a higher optical purity than the initial supersaturated solution.

### Example 3

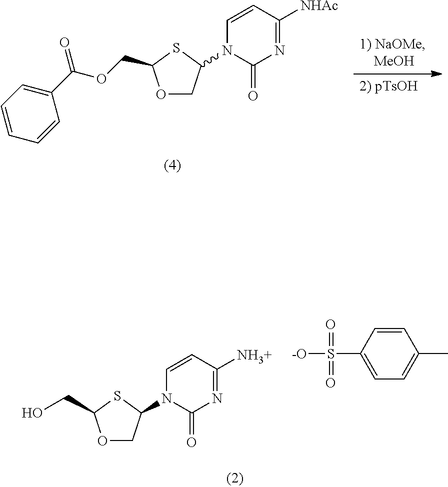

Sodium methoxide (0.1 eq.) was added in one portion to a methanol suspension of Compound (4) (1.0 eq, C/T=3.04/1, 95% ee, 96.6% purity) at room temperature. The reaction mixture was stirred for 2 hrs at room temperature. TLC analysis (Hexane/Et0Ac:1/9) showed the disappearance of starting material (Rf 0.10 (trans) and 0.16 (cis)) and the appearance of the more polar deprotected Compound (4) (Rf 0.00). The para-toluenesulfonic acid (1.14 eq.) was added to the solution in one portion at room temperature. The reaction mixture was allowed to stir at room temperature overnight then filtered. The solids were dried in vacuo to give Compound (2) salt as a white solid (62/1 cisltrans: 98% ee, 85% cis yield corrected).

### Example 4

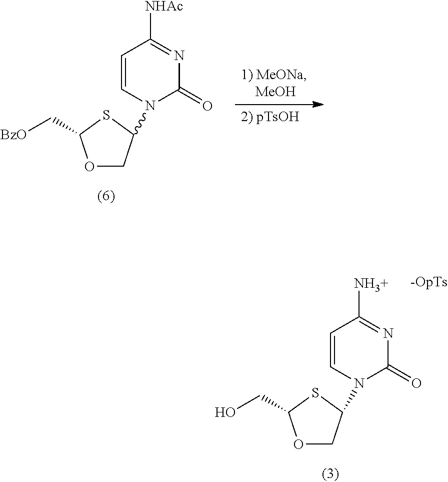

Sodium methoxide (0.1 eq.) was added in one portion to a methanol suspension of Compound (6) (1.0 eq., C/T=2.7/1, 95% ee, 96.0% purity) at room temperature. The reaction mixture was stirred for 2 hrs at room temperature. TLC analysis (Hexane/EtOAc:1/9) showed the disappearance of starting material (Rf 0.10 (trans) and 0.16 (cis)) and the appearance of the more polar deprotected Compound (4) (Rf 0.00). para-Toluenesulfonic acid (1.13 eq.) was added to the solution in one portion at room temperature. The reaction mixture was allowed to stir at room temperature overnight then filtered. The precipitate was dried in vacuo to give Compound (3) salt as a white solid (62/1 cisltrans: 99% ee, 86% cis yield corrected).

### Example 5

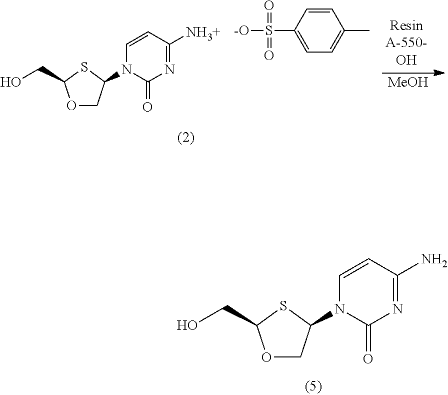

Compound (2) (9.76 mmoles, 1.0 eq.) was suspended in methanol at 40° C. Resin DOWEX 550A-OH (140% w/w) was added to the suspension in one portion at 40° C. The reaction mixture was allowed to stir at 40° C. for 2 hrs. The pH of the solution was checked to make sure that it's basic (pH≧7) and a sample was analyzed by ¹H NMR and showed the disappearance of para-toluenesulfonic acid. The reaction mixture was filtered. The resin was washed with methanol at 40° C. MeOH was distillated and the volume adjusted to 3 volumes. The solution was cooled and precipitation occurred. The suspension was stirred until no additional precipitation was observed then filtered. The solids were dried to give Compound (5) as a white solid (99.7% ee, 82% cis yield).

### Example 6

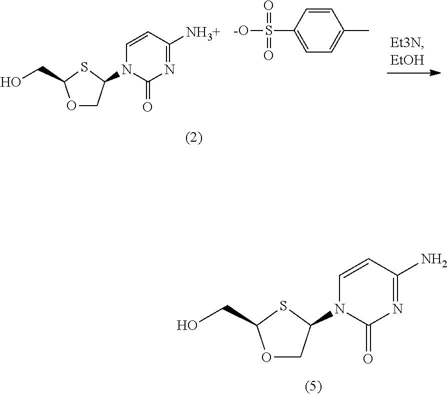

Compound (2) (99.1% ee, C/T=27/1), 6.23 mmoles of cis, 1.0 eq.) was suspended in ethanol at 25° C. Triethylamine (9.33 mmoles, 1.5 eq.) was added to the suspension in one portion at 25° C. The reaction mixture was heated at 40° C. and stirred for 1 hr at this temperature. The pH of the solution was checked to make sure that it's basic (pH≧7). The solution was cooled and precipitation occurred. The suspension was stirred until no additional precipitation was observed then filtered. The solids were washed with cold ethanol. The solids were dried to give Compound (5) as a white solid (99.4% ee, 80% cis yield).

### Example 7

Compound (5) was analysed on a C18 column (length 15 cm, diameter 4.6 mm, particle size 3 μm) using the following HPLC conditions:

Isocratic elution

Mobile Phase: 43% acetonitrile, 57% water+phosphoric acid 1/1000

Flow: 2 mL/min

Stop time: 10 min

Injection volume: 20 μL

Wavelength: 225 nm

Oven temperature: 40° C.

The amount of methyl tosylate and iso-propyl tosylate detected (in ppm) in each batch of Compound (5) is shown in Table 3

TABLE 3

| Item | Methyl tosylate | | Isopropyl tosylate | |
|---|---|---|---|---|
| LOQ | 0.1628 | μg/ml | 0.2431 | μg/ml |
| | 1.6 | ppm | 2.4 | ppm |
| LOD | 0.0651 | μg/ml | 0.0851 | μg/ml |
| | 0.65 | ppm | 0.85 | ppm |

| | | | |
|---|---|---|---|
| Batch 30-1600 | 0.1 | ppm | ND |
| Batch 30-1727 | 0.8 | ppm | ND |
| Batch 30-1739 | 0.4 | ppm | ND |
| Batch 30-1850 | 0.2 | ppm | ND |
| Batch 30-1875 | 0.5 | ppm | ND |
| Batch 30-2077 | 0.8 | ppm | ND |

LOQ is Limit of Quantification
LOD is Limit of Detection

### Example 8

Screening for a Conglomerate of cis 2′-deoxy-3′-oxa-4′-thiocytidine

The amine functionality of cis 2′-deoxy-3′-oxa-4′-thiocytidine was derivatized by salt formation with achiral acids. Four major groups of acids were screened to identify salts that may be candidates exhibiting conglomerate behavior:
Inorganic acids (e.g.: HCl, HBr, H₂SO₄, HBF₄);
Sulfonic (e.g.: methanesulfonic, benzenesulfonic, p-toluenesulfonic, p-hydroxytoluenesulfonic, sulfanilic, p-cholorobenezenesulfonic);
Substituted acetic acids (e.g.: glycolitic, chloro-, dichloro-, trichloroacetic); and
Polycarboxylic and oxy acids (e.g.: succinic, adipic, maleic, fumaric, citric, pyruvic).

In each case the salts of the racemic mixture and the single enantiomer were generated by reacting the nucleoside with an acid in water until the nucleoside was completely dissolved. The mixture was heated, if needed, until a clear solution was obtained. The salts were precipitated by vacuum concentration of the aqueous solution followed by the addition of isopropanol. The salt formation was confirmed ¹H NMR. In the case of HCl and HBr salts, a silver nitrate titration was performed. If a solid resulted an IR spectra was obtained.

A conglomerate compound crystallizes as a single enantiomer in the crystal lattice. This means that for a conglomerate compound the IR spectrum of the racemate (1:1 mixture of enantiomers) will be identical to that of the single enantiomer. Another characteristic of conglomerate behavior is that the racemate salt will have a melting point at least 25° C. lower than that of either single enantiomer salt.

For the screened salts, DSC data was obtained for each solid (see Table 4 below) and a binary melting point diagram was generated.

TABLE 4

| | Salt Formed? | | | DSC data (° C.) | | Possible |
|---|---|---|---|---|---|---|
| | | Single | IR | | Single | Conglomerate |
| Achiral Acid | Racemate | Enantiomer | Match? | Racemate | Enantiomer | Candidate? |
| HCl | YES | YES | NO | 137.8/229.2 | 139.1/221.5 | NO |
| HBr | YES | YES | NO | 228.8 | 217.9 | NO |
| H₂SO₄ | YES | YES | NO | 226.2 | 142.1/228.4 | NO |
| HBF₄ | YES | YES | NO | 150.5/211.5 | 218.3 | NO |
| Methanesulfonic | YES | YES | NO | 192.6 | 196.9 | NO |
| CH₃SO₃H | | | | | | |
| Benzenesulfonic | YES | YES | NO | 205.1 | 192.0 | NO |
| (BS) | | | | | | |
| C₆H₅SO₃H | | | | | | |
| p-ChloroBS | YES | YES | NO | 126.8 | 146.6 | NO |
| p-ClC₆H₄SO₃H | | | | | | |
| p-Toluenesulfonic | YES | YES | YES | 185.2 | 214 | YES |
| p-CH₃C₆H₄SO₃H | | | | | | |
| p-AminoBS | YES | YES | YES | 224.0 | 227.1 | YES |
| p-NH₂C₆H₄SO₃H | | | | | | |
| Glycolic | YES | YES | YES | 74.9/173.4 | 79.5/143.4 | YES |
| HOCH₂COOH | | | | | | |
| Maleic Cis- | YES | YES | YES | 172.0 | 166.9 | YES |
| HOOC(CH₂)₄COOH | | | | | | |

The DSC data confirmed the p-toluenesulfonate salt as a conglomerate, the melting point of the racemic salt was lowered by between 27.6 to 28.6° C. (186.6° C.) than that of the enantiomeric salt (see below). Solubility tests in water and methanol showed that the solubility of the racemic p-toluenesulfonate salt (ca. 13 mL/g) was significantly higher than that of the enantiomeric salt (ca. 25 mL/g). Similarly, in methanol the racemic p-toluenesulfonate salt had a solubility of 37 mL/g while the enantiomeric salt was 65 mL/g. The IR match was confirmed.

TABLE 5

| p-Toluenesulfonate | Melting point | ΔH |
|---|---|---|
| Salt | ° C. | J/g |
| Racemic | 186.6 | 100.2 |
| (−) enantiomer | 213.2 | 145.6* |
| (+) enantiomer | 214.2 | |

*corrected value

The other candidates were recrystallized from methanol/water/IPA and methanol/water mix. The results obtained for the racemic maleic salt are inconclusive. The IR spectrum matched and the melting point difference is significant (see below). The other candidates were confirmed as non-conglomerates.

TABLE 6

| | Melting point |
|---|---|
| Maleic Acid Salt | ° C. |
| Racemic | 172.3 |
| Single enantiomer | 238.8 |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for the preparation of single enantiomers of a compound of formula (B), in either the cis or trans configuration, or a pharmaceutically acceptable salt or ester thereof, said process comprising: 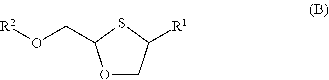
wherein
R¹ is pyrimidine base or a pharmaceutically acceptable derivative thereof;
R² is hydrogen, —C(O)—R³, or together with the oxygen atom to which it is attached forms an ester derived from a polyfunctional acid; and
R³ is hydrogen, straight or branched chain alkyl, alkoxyalkyl, aralkyl, aryloxyalkyl, aryl, substituted dihydropyridinyl, a sulphonate ester, a sulfate ester, an amino acid ester, a mono, di- or triphosphate esters;
said process comprising forming a conglomerate salt of racemic mixture or an enantiomerically enriched mixture of a compound of formula (B) with an acid wherein the resulting conglomerate salt has the following characteristics:
the IR spectrum of the salt of the racemic compound, a 1:1 mixture of (−) and (+) crystals, is identical to each of the single enantiomers, and
the salt of the racemic compound has a melting point lower than that of either single enantiomer; and resolving said mixture by crystallization.

2. A process according to claim 1, wherein the crystallization process is preferential crystallization.

3. A process according to claim 1, wherein the crystallization process is entrainment or cyclic entrainment.

4. A process according to claim 1, wherein
R¹ is selected from the following formulae: 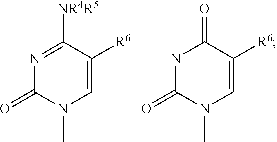
R⁴ and R⁵ are in each case independently H, straight, branched or cyclic C₁₋₆ alkyl, straight, branched or cyclic C₂₋₆ alkenyl, C₆₋₁₄ aryl, or 5-10 membered heteroaromatic ring containing 1-3 heteroatoms selected from O, N, and S; and
R⁶ is hydrogen, hydroxymethyl, trifluoromethyl, straight, branched or cyclic C₁₋₆ alkyl, straight, branched or cyclic C₂₋₆ alkenyl, bromine, chlorine, fluorine, or iodine.

5. A process according to claim 1, wherein R³ is hydrogen, straight or branched chain alkyl, alkoxyalkyl, aralkyl, aryloxyalkyl, aryl, substituted dihydropyridinyl, alkylsulphonyl, aralkylsulphonyl, a sulfate ester, an amino acid ester, and mono, di- or triphosphate esters, and pharmaceutically acceptable salts and esters thereof.

6. A process according to claim 5, wherein R³ is hydrogen, methyl, ethyl, n-propyl, t-butyl, n-butyl, methoxymethyl, benzyl, phenoxymethyl, phenyl, phenyl substituted by halogen, C₁₋₄ alkyl or C₁₋₄ alkoxy, N-methyldihydropyridinyl, methanesulphonyl, L-valyl or L-isoleucyl.

7. A process according to claim 6, wherein R³ is methyl, ethyl, n-propyl, t-butyl, n-butyl, methoxymethyl, benzyl, phenoxymethyl, phenyl, or phenyl substituted by halogen, C₁₋₄ alkyl or C₁₋₄ alkoxy.

8. A process according to claim 1, wherein R² is an ester derived from a dicarboxylic acid of the formula HO₂C(CH₂)ₙCO₂H where n is an integer of 1 to 10.

9. A process according to claim 1, wherein single enantiomers of formula (B) in the trans configuration are prepared.

10. A process according to claim 1, wherein single enantiomers of formula (B) in the cis configuration are prepared.

11. A process according to claim 1, wherein the single enantiomers of the conglomerate salt show a much lower solubility than the racemate of the conglomerate salt in polar solvents.

12. A process according to claim 1, wherein enantiomer separation of the enantiomeric mixture is performed by seeding a supersaturated solution of the conglomerate salt with the desired single enantiomer.

13. A process according to claim 1, wherein R¹ is cytosine or 5-fluorocytosine.

14. A process according to claim 13, wherein R¹ is cytosine.

15. A process according to claim 13, wherein R¹ is 5-fluorocytosine.

16. A process according to claim 1, wherein said acid is hydrochloric acid, hydrobromic acid, sulfuric acid, tetrafluoroboric acid, methanesulfonic acid, benzenesulfonic acid, para-toluenesulfonic acid, p-amino benzenesulfonic acid, p-chloro benzenesulfonic acid, p-hydroxy benzenesulfonic acid, chloroacetic acid, dichloroacetic acid, trichloroacetic acid, glycolic acid, pyruvic acid, succinic acid, adipic acid, maleic acid, fumaric acid, citric acid, or a mixture thereof.

17. A process according to claim 16, wherein said acid is para-toluenesulfonic acid, maleic acid or a mixture thereof

18. A process according to claim 1, wherein the conglomerate salt formed is cis 2′-deoxy-3′-oxa-4′-thiocytidine.

19. A process according to claim 1, wherein a conglomerate salt of cis 2′-deoxy-3′-oxa-4′-thiocytidine is formed in which the single enantiomers show a lower solubility than the racemate in polar solvents.

20. A process according to claim 19, wherein said conglomerate salt of cis 2′-deoxy-3′-oxa-4′-thiocytidine is the para-toluenesulfonic acid salt of 2′-deoxy-3′-oxa-4′-thiocytidine having an eutectic point between about 185° C. and 187° C.

21. A process according to claim 19, wherein said conglomerate salt of cis 2′-deoxy-3′-oxa-4′-thiocytidine is the malic salt of 2′-deoxy-3′-oxa-4′-thiocytidine having an eutectic point between about 171° C. and 173° C.

22. A process according to claim 1, wherein the single enantiomer further comprises a second isomer of a compound of formula (B) in an amount equal to or less than 1%.

23. A process for resolving cis-2-hydroxymethyl-4-(cytosin-1′-yl)-1,3-oxathiolane or derivatives or salts thereof comprising:
a) reacting said cis-2-hydroxymethyl-4-(cytosin-1′-yl)-1,3-oxathiolane with an achiral acid to produce cis-2-hydroxymethyl-4-(cytosin-1′-yl)-1,3-oxathiolane•achiral acid salt;
b) preparing a solution of cis-2-hydroxymethyl-4-(cytosin-1′-yl)-1,3-oxathiolane•achiral acid salt having an enantiomeric excess greater than zero;
c) adding to said solution an amount of (+) or (−)-cis-2-hydroxymethyl-4-(cytosin-1′-yl)-1,3-oxathiolane•achiral acid salt sufficient to initiate crystallization;
d) recovering substantially one of said (+) or (−)-cis-2-hydroxymethyl-4-(cytosin-1′-yl)-1,3-oxathiolane•achiral acid salt; and
e) converting said (+) or (−)- cis-2-hydroxymethyl-4-(cyto sin-1′-yl)- 1,3-oxathiolane•achiral acid salt into said (+) or (−)-cis-2-hydroxymethyl-4-(cytosin-1′-yl)-1,3-oxathiolane or salts.

24. A process according to claim 23, wherein said achiral acid is hydrochloric acid, hydrobromic acid, sulfuric acid, tetrafluoroboric acid, methanesulfonic acid, benzenesulfonic acid, para-toluenesulfonic acid, p-amino benzenesulfonic acid, p-chloro benzenesulfonic acid, p-hydroxy benzenesulfonic acid, chloroacetic acid, dichloroacetic acid, trichloroacetic acid, glycolic acid, pyruvic acid, succinic acid, adipic acid, maleic acid, fumaric acid, citric acid, or a mixture thereof.

25. A process according to claim 23, wherein said acid is para-toluenesulfonic acid.

26. The conglomerate salt of cis 2′-deoxy-3′-oxa-4′-thiocytidine is the para-toluenesulfonic acid salt of 2′-deoxy-3′-oxa-4′-thiocytidine having an eutectic point between about 185° C. and 187° C.

27. The conglomerate salt of cis 2′-deoxy-3′-oxa-4′-thiocytidine is the maleic salt of 2′-deoxy-3′-oxa-4′-thiocytidine having an eutectic point between about 171° C. and 173° C.

28. A process for the preparation of a single enantiomer of formula (B) or a pharmaceutically acceptable salt or ester thereof, wherein the enantiomer comprises methyl tosylate in an amount equal to or less than 2 ppm, the process comprising the steps of:
(a) forming a conglomerate salt of a racemic mixture or an enantiomerically enriched mixture of a compound of formula (B) with a tosic acid;
(b) obtaining an enantiomerically enriched mixture of the salts of the enantiomers by crystallization; and
(c) obtaining the free base of the enantiomerically enriched mixture.

29. A process according to claim 28, wherein the tosic acid is para-toluenesulfonic acid.

30. A process according to claim 28, wherein the compound of formula (B) is 2′-deoxy-3′-oxa-4′-thiocytidine.

31. A process according to claim 28, wherein the enantiomer is in the cis configuration.

32. A process according to claim 28, wherein the enantiomer further comprises a second isomer of a compound of formula (B) in an amount equal to or less than 1%.

33. A composition comprising a single enantiomer of 2′-deoxy-3′-oxa-4′-thiocytidine or a pharmaceutically acceptable salt or ester thereof, wherein the enantiomer comprises methyl tosylate in an amount equal to or less than 2 ppm.

34. A composition according to claim 33, wherein the enantiomer is in the cis configuration.
